(19) United States
(12) Patent Application Publication
(10) Pub. No.: US 2026/0207145 A1
(43) Pub. Date: Jul. 23, 2026

(54) Method And Device For Indicating The Risk Of Ankle Injury

(71) Applicant: Uovodoro Informatikai Korlátolt Felelosségu Társaság, Onga (HU)

(72) Inventor: Daniel Molitorisz, Budaors (HU)

(21) Appl. No.: 19/138,834

(22) PCT Filed: Sep. 18, 2023
(86) PCT No.: PCT/HU2023/050060

§ 371 Date: Jun. 13, 2025

(30) Foreign Application Priority Data
Dec. 14, 2022 (HU) P2200481

(51) Int. Cl. A61B 5/00; A61B 5/103

(52) U.S. Cl. CPC A61B 5/7275; A61B 5/1038; A61B 5/6807

(57) ABSTRACT

The invention is a method and a device for indicating a risk of supination ankle injury, wherein a first and a second signal value sequence (P₁, P₂) are provided by a first and a second pressure force sensor (21, 22) arranged in a first and in a second insole region (14, 15) of insole (10) placed in a shoe, and, during a foot-ground interaction process
a first aggregate value is calculated utilising first signal value sequence (P₁) values within a first time interval determined by a value of the first signal value sequence (P₁) indicating an appearance of a pressure force (P₁₀) and a value of the first signal value sequence (P₁) indicating maximum pressure force (P₁ₘₐₓ),
a second aggregate value is calculated utilising second signal value sequence (P₂) values within a second time interval determined by a value of the second signal value sequence (P₂) indicating an appearance of a pressure force (P₂₀) and a value (P_{2P1max}) of the second signal value sequence (P₂) taken at the time instant corresponding to the time instant of the value (P₁ₘₐₓ) of the first signal value sequence (P₁) indicating maximum pressure force, and
determining an existence of a risk of supination ankle injury, or an extent of said risk, based on a comparison of the first aggregate value and the second aggregate value.

### TECHNICAL FIELD

The invention relates to a method and device for indicating (signalling) the risk of ankle injury. More particularly, the invention is a solution aimed at preventing the type of injury that involves ankle sprain, i.e., in medical terms, the supination trauma and distortion of the ankle. During this injury, the ankle (the talofibular and talotarsal joints) is rotated and bent inward, simultaneously with bending the tarsus.

### DESCRIPTION OF PRIOR ART

The most frequently occurring lower-limb injuries during everyday and sports activities are the injuries of the knee and the ankle. It is a common trait of both injury types that the age groups most affected by them are the most physically active age groups, i.e., teenagers and young adults. It also frequently happens that after an ankle injury the knee also becomes injured, or vice versa.

The frequency of occurrence of supination ankle injury is also highest in the age group of 10-18 years, but it can occur in all age groups. It is also the most frequently occurring lower-limb trauma in sporting activities, i.e., 85% of ankle injuries are of the supination type. Repeated injuries lead to the instability of the talocrural joint, to joint damage, and pain during movement. These factors may cause, both in themselves and in combination with each other, a limited range of motion, unsteady gait, and a reduction of the speed of motion. All in all, this results in a reduction of the physical performance of the lower limbs, and, consequently, deteriorated sporting performance.

Furthermore, following a supination ankle injury, the risk of repeated ankle injuries significantly increases, together with the risk of consequential knee injuries.

The biomechanics of supination ankle injury is well known, but the movements performed before and leading up to the supination injury have not hitherto been brought to the focus of analysis. The reason for this is the great variety and complexity of movements, especially of sports movements. The load placed on the lower limbs-among others, the sports-specific loads on the ankle-differs from sport to sport. The physical and biomechanical characteristics of sports-specific steps, jumps, and landings vary on a wide scale.

In scientific literature, the laxity of the ankle's ligament system and its possible earlier injuries are typically mentioned as the conditions indicating the risk of ankle injury. Other studies interpret the analysis of predictive factors as the interconnected or independent understanding of bodily characteristics and postural instability. The terms micro- and macro-instability have been applied for the acquired movement function limitation of the ankle that occurs after an earlier injury. In the case of micro-instability, pain occurs in a characteristic region of the lateral ankle if the ankle is loaded—the arthroscopy finding indicates the thickening of the sATFL—, while in the case of macro-instability, the talus becomes abnormally movable in the ankle fork.

In the technical field there exists a need for a method and device for indicating the risk of ankle injury, and in particular for a technical solution enabling the prevention of supination-type injuries of the ankle.

Technical solutions for gait or movement analysis comprising sensors integrated in a shoe or insole are disclosed for example in the following documents: US 2012/0253234 A1, DE 10 2011 012 458 A1, US 2013/0213145 A1, US 2015/0182844 A1, US 2017/055880 A1, US 2017/0116869 A1, US 2016/0345865 A1, US 2017/0188950 A1, US 2019/0099113 A1, US 2019/0175107 A1, US 2018/0049670 A1, US 2020/0021896 A1, US 2018/0333078 A1, U.S. Pat. No. 10,595,749 B1, US 2019/0099123 A1, WO 2019/086997 A2, WO 2019/175899 A1, US 2020/0000375 A1, US 2021/0298413 A1.

In US 2008/0216593 A1 a gait toe-off promoting device is disclosed that comprises a first sensor located proximate the ball of a foot and configured to sense the force acting on the ball of the foot during a gait cycle, a second sensor located proximate a pressure receiving surface of a toe of the foot, expediently the big toe, and configured to measure the force acting on the toe of the foot during the gait cycle, a control centre configured to process the signals received from the first and second sensors, and a feedback mechanism controlled by the control centre and configured to notify the individual of improper toe-off during the gait cycle, thus making the individual aware of the need to correct future gait cycles. The document is aimed at the correction of improper gait caused by digitus malleus/bunion. This technical solution applies the maximum values of the pressure force, and the differences between them, as a basis for the feedback.

The prior art technical solutions are not able to effectively indicate the risk of supination ankle injury to a user.

### DISCLOSURE OF THE INVENTION

The object of the invention is to provide a method and a device that are able to indicate the risk of supination ankle injury effectively, and, if possible, with low computational demand. The invention is based on the recognition below.

The effective prevention of injury necessitates predictive prevention measures, which include warning the user when a movement sequence is performed that causes an increased risk of injury. Distortion injuries of the ankle are preceded by a critical moment wherein a loss of balance occurs. This loss of balance occurs in the sole of the foot area that constitutes a single functional unit together with the ankle, providing support during movement.

Typically, the following sequence of movements leads to the critical movement: During walking and running gait, the stance phase of the gait starts with the initial contact between the heel and the ground, continuing along the lateral edge of the foot, and concluding on the metatarsi IV-V, the ball of the foot portions of the metatarsophalangeal joints, and to a varying degree, on the MP joint portion of the metatarsus I. Modelling the movements of the stance phase, two nearly mutually perpendicular curves can be identified along which the movements occur: a curve nearly parallel to the longitudinal arch of the foot, and a curve connecting the lateral edge of the sole of the foot and the ball of the foot near the hallux MP joint. Less often during straight-line movement but more frequently during evasive manoeuvres and turns (movements along a circular line), the stance phase may lack a pronounced placement of weight on the hallux MP area or on the ball of the foot near the big toe. In such cases, the projection of the dynamic force line is a circular arc.

The critical moment:

According to the recognition of the invention, in the critical moment a well-defined point of the weight-bearing area of the sole is lost; this point is the same in the case of all movement types, i.e., it is independent of the movement performed and thus it is not sports-specific. Namely, this critical event is the lifting of the big toe—hallux—from the ground surface. Lifting of this point of the weight-bearing area occurs in the (single-limb) stance phase of the gait (i.e., in any of the “load response”−“midstance”-“terminal stance” phases), or in any other corresponding weight-loading ground-contact position. Due to the lifting of the weight-bearing ground-contact point of the sole, the foot and the lateral ankle (which forms functional unit with the foot) are brought into an unstable balance position. In this position it is not in a “steady state”, i.e., it either regains its original stable support position or a loss of balance and consequential injury occurs. The unstable balance position is preceded by a pressure force reduction, without which no ground-contact sole point lifting can occur. In this sense, the reduction or loss of pressure force on the big toe in the stance phase has a predictive role from the aspect of a potential ankle injury involving ankle “sprain”.

The invention also includes the recognition that the irreversible tilting of the ankle that inevitably leads to an injury occurs in relation to this well-defined region of the sole of the foot lifting from the ground surface; it has been provable with video footage showing ankle injuries with ankle sprain occurring during sports.

The loss of the ground-contact point of the big toe in itself does not amount to ankle injury. In this sense, this event is a necessary condition of injury but is not sufficient in itself, i.e., it highly increases the risk of a future potential ankle injury. It has been proven with dynamic tests undertaken with the full body weight that lifting of the big toe from the ground-contact surface leads to a loss of balance but does not result in ankle injury. According to physical laws, the reduction of pressure force is required for the lifting of the big toe, so pressure force reduction has predictive value for big toe lifting. During movement in the stance phase of the gait, the lifting of the big toe inevitably leads to a loss of balance. After losing balance, either a compensating sequence of movements is carried out to regain balance, or an injury occurs.

In sum, it can be maintained that pressure reduction under the big toe ground-contact point in the stance phase of the gait is a predictive factor for ankle injury.

The sequence of events leading to the injury is the following:
reduction or loss of pressure on the contact point of the big toe, or lifting of the big toe from the ground surface,
assuming an unstable balance point-loss of balance
ankle injury or regaining of stable balance.

Based on assessing the events in this process and their sequence, it can be maintained that sports-specific movements are not necessary for a loss of balance to occur. This process is the same for all distortion ankle injuries occurring in sports and during sports movements.

Following the recognition above, the invention was also motivated by the additional recognition that processing the maximum pressure force values according to US 2008/0216593 A1 in itself cannot be successfully applied for the inventive prediction because these values do not contain temporal information; for providing appropriate predictions, the temporal relationship between force interactions must also be taken into account.

The objects according to the invention have been achieved by providing the method according to claim 1, the device according to claim 13, and the shoe comprising the device according to claim 21. Preferred embodiments of the invention are defined in the dependent claims.

In the experiments it has been found that by applying sensors arranged in measurement regions according to the invention and considering the occurring forces by aggregating them in appropriate time intervals an effective prediction can be provided. Based on the aggregates of the forces in a time interval such a quantitative measure can be calculated which is proportional to the amount of muscle work performed, and it is precisely the insufficient amount of (location-specific) muscle work which may lead to situations with increased risk of injury. For making the appropriate calculations it is also necessary that the aggregation is performed in temporally synchronised time intervals, which can be most expediently and simply implemented by setting identical start and end time instants for the time intervals. This is because the appropriate support of pressure of the big toe is extremely important until the lateral portion of the ball of the foot reaches maximum pressure-after that, i.e., in the subsequent movement phase, this pressure area has continually decreasing importance for injury prevention.

### BRIEF DESCRIPTION OF THE DRAWINGS

Preferred embodiments of the invention are described below by way of example with reference to the following drawings, where

FIG. 1 is a schematic view of a preferred embodiment of the device according to the invention,

FIG. 2A illustrates measurement regions and insole regions adapted for accommodating the sensors according to the invention,

FIG. 2B illustrates insole regions adapted for accommodating the sensors according to a particularly preferred embodiment,

FIG. 3A shows pressure maps of non-instructed step phases,

FIG. 3B shows pressure maps of instructed step phases,

FIG. 4A shows diagrams of first signal value sequences provided by a first pressure force sensor in the case of instructed and non-instructed steps taken with the left foot, indicating the regions applied for signal evaluation according to the invention,

FIG. 4B shows, on a common time axis, diagrams of signal value sequences provided by both pressure force sensors in the case of instructed and non-instructed steps taken with the left foot, indicating the regions applied for signal evaluation according to the invention,

FIG. 5A shows diagrams of first signal value sequences provided by the first pressure force sensor in the case of instructed and non-instructed running steps taken with the left foot, indicating the regions applied for signal evaluation according to the invention, and

FIG. 5B shows diagrams of signal value sequences provided by both pressure force sensors in the case of instructed and non-instructed running steps taken with the left foot, indicating the regions applied for signal evaluation according to the invention.

### MODES FOR CARRYING OUT THE INVENTION

In the course of the measurement process, pressure values and the time intervals required for the buildup of the pressure values are detected at two different regions of the sole of the foot, during the initial contact, midstance, and terminal stance phases of the step or another foot-ground interaction process. By processing the measured pressure force values according to the invention, it is possible to give a clear indication of steps, running steps, jumps, or other foot-ground interaction processes causing a risk of supination-type ankle injuries. Measuring the foot sole pressure or pressure change is expediently implemented in the foot sole region, but it is also possible to measure it in the dorsum-of-foot region.

Therefore, sensors adapted for detecting mechanical pressure force in the sole or dorsum-of-foot side of the foot are arranged on or within the shoe-insertable insole according to the invention in two measurement regions. With the help of the signals received from these measurement regions a dynamic digital pressure map is generated in the initial contact, midstance, and terminal stance phases of the step.

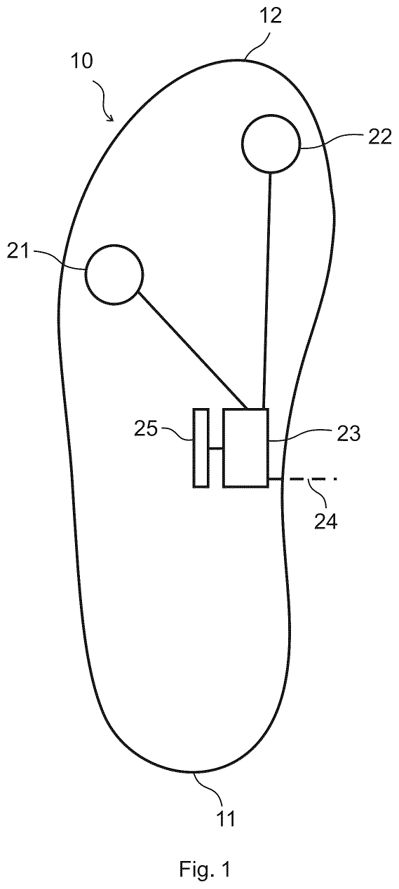

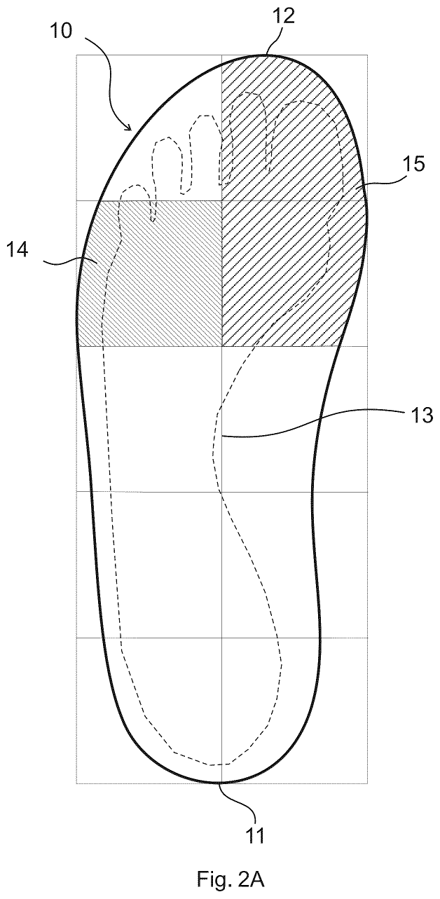

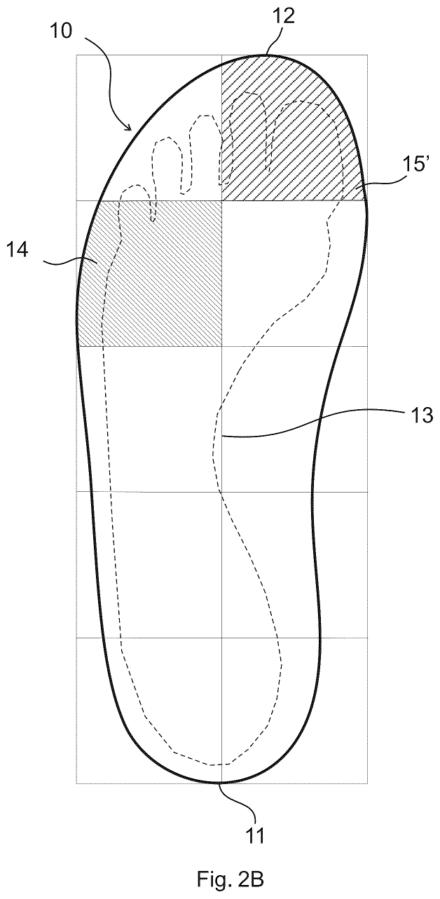

FIG. 1 shows a schematic view of a preferred embodiment of the device for indicating the risk of supination ankle injury according to the invention. The device comprises a shoe-insertable insole 10 having a posterior end 11 and an anterior end 12, and a respective sensor in both regions defined according to FIG. 2A, more preferably according to FIG. 2B.

A first pressure force sensor 21 is arranged in a first insole region 14 between three-fifths and four-fifths of a longitudinal expanse of the insole extending from the posterior end 11 to the anterior end 12, and laterally outward from a centreline 13 of said longitudinal expanse in a transversal direction perpendicular to the longitudinal direction. A second pressure force sensor 22 is also arranged in a second insole region 15 between three-fifths and the anterior end 12 of a longitudinal expanse of the insole extending from the posterior end 11 to the anterior end 12, and laterally inward from the centreline 13 of said longitudinal expanse in a transversal direction perpendicular to the longitudinal direction.

According to the shoesize-independent definition given above, the first and second insole regions 14 and 15 are suited for the purposes of the invention because they include those lateral and medial regions of the sole of the foot which can provide signals allowing effective prevention. Namely, in FIGS. 2A and 2B the first insole region 14 including the lateral region of the balls of the foot under the metatarsophalangeal joint V is indicated by hatching to the left of the centreline 13, while the second insole region 15 including the region of big toe and the ball of the foot under the big toe, or a preferable subregion 15′ thereof, is indicated by hatching in FIGS. 2A and 2B to the right of the centreline 13. These two regions are clearly sufficient for obtaining signal streams or signal value sequences allowing the recognition of motion processes leading to ankle injury.

To use an alternative wording, the first and second insole regions 14 and 15 are obtained by uniformly dividing a bounding rectangle of the insole 10 laterally to 2 segments and longitudinally to 5 segments, and thus the rectangle is divided into such quadrangles (i.e., smaller rectangles) that have a width corresponding to half of the transversal bounding width of the insole, and a length corresponding to fifth of the bounding length of the insole 10. The first insole region 14 extends over the fourth outer quadrangle (as counted from the heel), while the second insole region 15 extends over the fourth and fifth inner quadrangles (as counted from the heel). A preferred subregion 15′ of the second insole region 15 extends only over the fifth medial quadrangle (as counted from the heel). The bounding rectangle is preferably oriented in such a way that the line connecting the support point of the foot at the calcaneus and the support point on the insole of the terminal section of the second toe (phalanx distalis dig. II pedis) is parallel with the longitudinal side of the rectangle.

The experiments have shown that the invention is able to give adequate predictions with a second pressure force sensor 22 positioned anywhere in the second insole region 15, because the pressure force signals detectable in this region give an adequate picture of the loads on the big toe. In a preferred embodiment of the invention, however, a second pressure force sensor 22 arranged in the subregion 15′ of the second insole region 15 that is between four-fifths and the anterior end 12 of a longitudinal expanse extending from the posterior end 11 of the insole 10 to the anterior end 12 of the insole 10 is applied. In such an embodiment, as can be seen in FIG. 1, the second pressure force sensor 22 is arranged under the pad of the big toe, or in its vicinity, and is thus able to detect the pressure conditions under the big toe in the most direct way possible and allows for a more focused measurement. For the sake of simplicity, hereinafter reference is made everywhere to the second insole region 15 in general.

There are a number of different feasible technical solutions for detecting and measuring the mechanical pressure forces arising in the measurement regions during gait:
pressure or force sensors, for example based on the electric signal caused by a varying resistance,
based on the principle of the piezoelectric effect,
direct analysis of micro-camera video recordings,
analysis of heatmaps, etc.

In a preferred embodiment of the invention, the variation of pressure force is measured through measuring resistance change, by means of a pressure force-sensitive electric resistor. The electrical resistance values obtained through measuring the pressure force-variable resistance can be depicted along a time axis as a function. In the context of this invention, the terms “force” and “pressure” are used equivalently with the expression “pressure force”, because they arise due to the same physical reason, as a result of an indirect contact with the ground during steps and other movements, and therefore they can all be applied for the purposes of the invention in an identical manner.

The first pressure force sensor 21 and/or the second pressure force sensor 22 are preferably affixed onto the insole 10 or is fixed within the insole 10. 13. Such a solution is also conceivable wherein the insole 10 also has a dorsum-of-foot portion, for example configured like a strap (or band) of a sandal (or slider), with the first pressure force sensor 21 and/or the second pressure force sensor 22 being preferably affixed on the dorsum-of-foot portion or being fixed within the dorsum-of-foot portion; the insole regions 14 and 15 are to be interpreted in a top plan view also in such cases. Most preferably, the pressure force sensors 21 and 22 are embedded in the material of the insole 10 and are not in direct contact with the foot.

As depicted in FIG. 1, the device according to the invention further comprises a control unit 23 that is adapted for receiving and processing a first signal value sequence provided by the first pressure force sensor 21 and a second signal value sequence provided by the second pressure force sensor 22 and is programmed to carry out the method that will be described in detail below. As can be seen in FIG. 1, the control unit 23 is preferably arranged within the insole 10. Such an embodiment is also conceivable wherein the control unit 23 is at least partially implemented as an external unit, I.e., it is arranged partially or entirely outside the insole 10; for example, it is implemented as a separately programmed microprocessor unit or an application running on a mobile phone. In extreme cases only the pressure force sensors 21, 22 are arranged within the insole such that they are in communication with an external control unit 23. In such cases, therefore, the device according to the invention further comprises a wired or wireless communication channel 24 connecting the external unit to the other components of the device.

As illustrated in FIG. 1, the existence of a risk of supination ankle injury or the extent of said risk surpassing a threshold value can be indicated to a user wearing the shoe provided with the insole 10 by an indicator device 25 arranged within the insole 10. The indicator device 25 can of course also be arranged on the user's body or in the vicinity of the user, and can preferably be a haptic, audio, or visual indicator device.

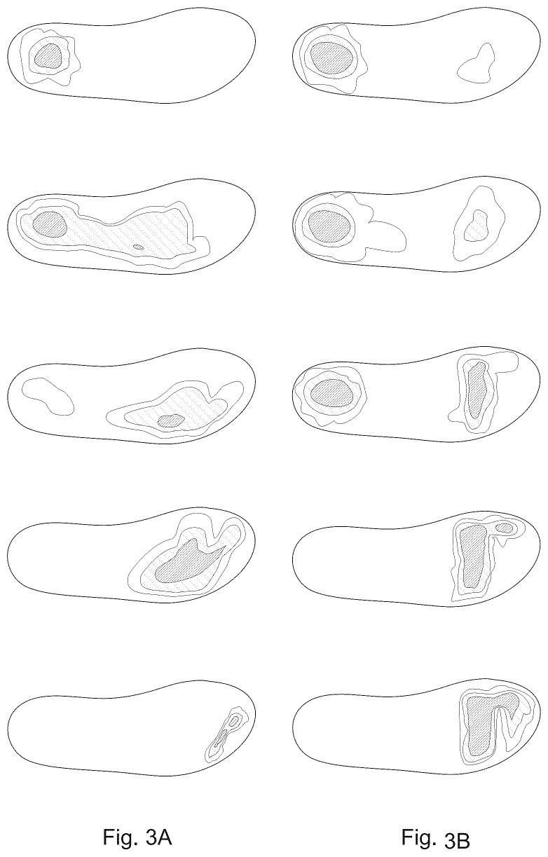

FIGS. 3A and 3B show the pressure maps of non-instructed and instructed step phases recorded during an experiment. In the figures there can be observed how the pressure force builds up and decreases during the different step phases. The instructed step phases illustrate that, based on the indications of the device or following the instructions of an expert, the subject puts more load (more of his/her body weight) on the big toe, thereby reducing the risk of ankle injury.

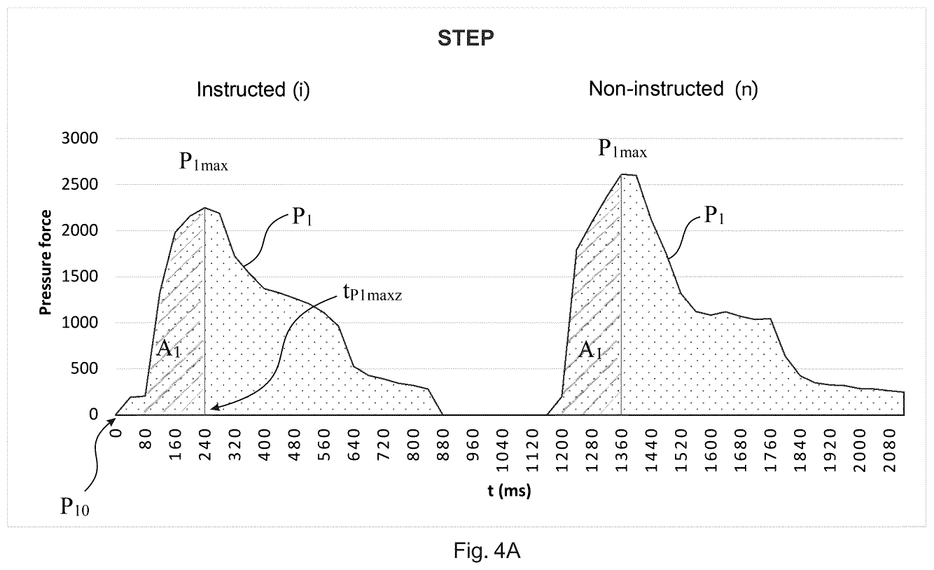

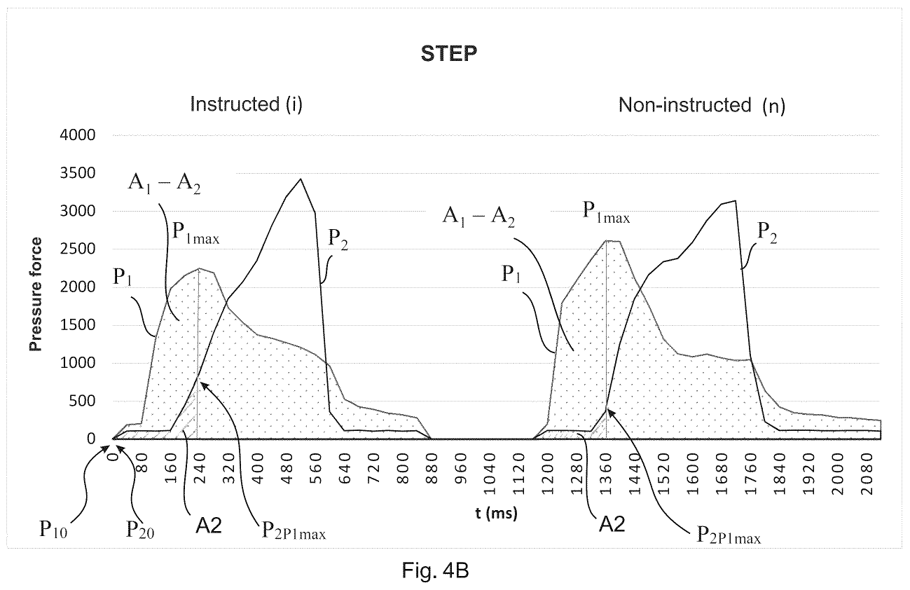

FIG. 4A shows diagrams of first signal value sequences P₁ supplied by the first pressure force sensor 21 in the case of instructed and non-instructed steps taken with the left foot, indicating the area values A₁, i.e., area measures or area sizes applied for an exemplary signal evaluation process according to the invention. The first signal value sequence P₁ supplied by the first pressure force sensor 21 is received applying a given signal sample rate that is preferably in the range of 25-100 Hz. The second signal value sequence P₂ that is supplied by the second pressure force sensor 22 and is shown in FIG. 4B is also received applying the same signal sample rate. In this figure and in the following figures, time is indicated on the horizontal axis of time diagrams utilising a ms scale; i.e., during signal processing, time is expediently measured in milliseconds.

A great advantage of the invention is that instead of applying the concrete resistance values or other values detected by the pressure force sensors 21 and 22, processing is performed by comparing aggregates (for example, area values) calculated therefrom, and thanks to that, calibration of the pressure force sensors 21 and 22 is not required. Therefore, the pressure force can be indicated on the vertical axes utilising an arbitrary scale; for example, the vertical axis can show N, Ohm, Pa values or other values proportional to the pressure force.

According to the invention, the following steps are carried out in the course of a foot-ground interaction process, which preferably forms a part of a walking, running, or jumping process, or a sporting activity.

We wait until a pressure force appears in the first signal value sequence P₁; at a value P₁₀ corresponding to this instant a first time interval is started, wherein a first aggregate value is calculated from the values of the first signal value sequence P₁, i.e., for example by calculating the area under the curve of the signal function or diagram a first area value A₁ is calculated. The first time interval ends with a value P₁ₘₐₓ of the first signal value sequence P₁ indicating maximum pressure force. Preferably, if the values of the signal value sequence P₁ reach the value P₁ₘₐₓ corresponding to the maximum pressure force more than once, then the time instant corresponding to the last of these values P_{1maxz} is considered as the endpoint of the first time interval. The denotations P₁ₘₐₓ and P₁ₘₐₓ z and the denotations t_{P1max} and t_{P1maxz} corresponding to the time instants thereof will hereinafter be used interchangeably; it always depends on the given measurement which of them is to be applied in the method.

Calculation of the first aggregate value can be implemented for example by the simple summation of the values of the first signal value sequence P₁ in the first time interval, or a calculation including such summation, which allows for very simple calculation and requires low computational capacity. Calculation of the first aggregate value may also comprise calculating the area under the function curve or signal diagram, which calculation essentially amounts to temporal integration, and can be implemented applying any suitable mathematical method, for example bar chart area calculation, trapezoid area calculation, or polynomial or other interpolation methods. In addition to that, other such suitable mathematical calculation methods can of course also be applied which yield a result that is characteristic of the totality of the values measured within the first time interval, for example calculating any type of average, etc. For calculating the first aggregate value preferably all the values of the first signal value sequence P₁ within the first time interval are utilised; however, such solutions are also possible wherein only certain values selected therefrom are applied, for example values are selected/omitted at regular intervals, or values that can be considered as measurement errors are disregarded.

FIG. 4A clearly illustrates the difference in pressure force buildup between instructed and non-instructed steps. In the case of instructed steps, more load is transferred to the big toe, so the first signal value sequence P₁ has a lower value P₁ₘₐₓ indicating maximum pressure force.

During the method, a first time interval can expediently be calculated that starts with a time instant t_{P1>0} corresponding to an appearance of the first P₁>0 pressure force value measured in the first insole region 14 and ends with a time instant t_{P1maxz} corresponding to a value P₁ₘₐₓ indicating maximum pressure force.

$$Δ {t}_{P 1 maxz} = {t}_{P 1 maxz} - {t}_{P 1 > 0}$$

In this first time interval, the pressure force values P₁ₘₐₓ≥P₁≥0 that can be detected up to the time instant t_{P1maxz} are measured in the first insole region 14.

FIG. 4B shows, on a common time axis, diagrams of signal value sequences P₁, P₂ provided by both pressure force sensors 21, 22 in the case of instructed and non-instructed steps taken with the left foot, indicating area values A₁, A₂ (i.e., area sizes) of the regions applied for signal evaluation. As can be seen in the figure, a second aggregate value is calculated utilising second signal value sequence P₂ values within a second time interval determined by a value P₂₀ of the second signal value sequence P₂ indicating an appearance of a pressure force and a value P₂P₁ₘₐₓ of the second signal value sequence P₂ taken at the time instant corresponding to the time instant of the value P₁ₘₐₓ of the first signal value sequence P₁ indicating maximum pressure force, for example, calculating the area under the signal function or signal diagram of the second signal value sequence P₂ and thus determining a second area value A₂.

The second aggregate value can be most preferably calculated in an identical manner to the calculation of the first aggregate value, so the description included above in relation to calculating the first aggregate value also applies to the calculation of the second aggregate value. Simple summation can be an expedient choice here as well, and, in case the two signal value sequences P₁, P₂ have identical sample rate, then this summation yields directly comparable results. However, such solutions are also conceivable wherein the two calculations are different to a greater or lesser extent, which difference is duly taken into account when the first aggregate value is compared with the second aggregate value. If, for example, the signal value sequences P₁, P₂ have different sample rates, then it can be expedient to apply temporal integration, which two integral calculations will have different time steps.

The time instant corresponding to the first signal value sequence P₁ value P₁ₘₐₓ indicating maximum pressure force is most preferably the time instant of the value P₁ₘₐₓ indicating maximum pressure force, which can easily be implemented with synchronous signal polling. It may happen, however, that the pressure force sensors 21, 22 are not polled synchronously or are polled not entirely synchronously, in which case the corresponding time instant will be the sample instant of the second signal value sequence P₂ that is closest to the time instant of the value P₁ₘₐₓ indicating maximum pressure force.

FIG. 4B shows a case wherein in the first signal value sequence P₁ the value P₁₀ indicating the appearance of a pressure force and the value P₂₀ indicating the appearance of a pressure force of the second signal value sequence P₂ occur at the same time instant, or at time instants with no visible time shift between them; however, the temporal difference between these two values depends on several factors. The solution according to the invention gives a satisfactory result in all circumstances, for example in the case of different movement forms or different anatomical or shoe parameters. The signal value sequences P₁ and P₂ preferably consist of pressure force values cleaned of measurement noise, including the values P₁₀ and P₂₀ indicating the appearance of pressure force.

Therefore, the pressure force values P₂≥0 that can be detected in the second insole region 15 up to the time instant t_{P1maxz} are also measured. Optionally, the time elapsed between the (temporarily) first pressure force value appearing in the first insole region 14 and the temporarily first pressure force value appearing in the second insole region 15 can also be calculated:

$$Δ {t}_{P 1 > 0 \to P 2 > 0} = {t}_{P 2 > 0} - {t}_{P 1 > 0}$$

Furthermore, the time interval between the time instant corresponding to the first and last pressure values (preferably cleaned of noise) appearing under the foot pad of the big toe (second insole region 15, values of the signal value sequence P₂) and the last appearance of the maximum value of the signal value sequence P₁ can also be calculated for each step.

According to the invention, the existence of a risk of supination ankle injury or the extent of said risk is determined based on the comparison of the first aggregate value, for example the area value A₁ and the second aggregate value, for example the area value A₂. Therefore, there are two different aspects in which the invention requires the smallest possible computational capacity for fulfilling its objectives. Namely, on the one hand, values are aggregated-applying for example area calculation-only up to the value P₁ₘₐₓ indicating maximum pressure force of the first signal value sequence P₁, and on the other hand, following the aggregation the calculation requires only the aggregate values, for example the first area value A₁ and the second area value A₂, which amounts to the simplest possible calculation.

Therefore, in a step time window preferably an area A₁ can be calculated, which is obtained as the area under the function curve or diagram in case the pressure force values of the first signal value sequence P₁ are plotted as a function or diagram along a time axis. For example, in the case of a function curve, the area can be obtained according to the formula of trapezoid area calculation:

$${A}_{1} = {\sum }_{P 1 = 0}^{tp 1 maxz - 1} f ( P 1 t ) * Δ {t}_{P 1 maxz}$$

Furthermore, in the step's time window preferably an area A₂ can also be calculated, which is obtained as the area under the function curve or diagram in case the pressure force values of the signal value sequence P₂ are plotted as a function or diagram along a time axis. For example, in the case of a function curve, the area can be obtained according to the formula of trapezoid area calculation:

$${A}_{2} = {\sum }_{P 2 = 0}^{tp 1 maxz - 1} f ( P 2 t ) * Δ {t}_{P 1 maxz}$$

In the following, the comparison of the aggregate values will be described in relation to comparing area values, but of course these preferred comparison options can be obtained in the case of other aggregation options. The comparison of the first area value A₁ and the second area value A₂ can be implemented in various ways. The existence of a risk of supination ankle injury, or the extent of said risk can be determined based on the ratio and/or difference of the first area value A₁ and the second area value A₂. In a preferred embodiment, an exposure risk value is determined by subtracting the second area value A₂ from the first area value A₁, and dividing the difference thus obtained by the first area value A₁, determining an existence of a risk of supination ankle injury, or the extent of said risk, based on the exposure risk value calculated in this manner. Optionally, a protection value is determined by dividing the second area value A₂ by the first area value A₁, and the existence of a risk of supination ankle injury, or the extent of said risk, is determined based on the exposure risk value calculated in this manner. The combination of the above, or other comparison methods can of course also be applied.

For example, the following ratio can be calculated: A₂:A₁

This ratio is typically smaller than 1, values approaching 1 give the extent of protection in percentage form. In the case of values greater than 1, the load placed on the big toe is of such magnitude that a supination ankle injury will not occur.

The difference between the areas at the time instant t_{P1maxz} or thereafter can also be calculated:

$${A}_{1} - {A}_{2} = {\sum }_{P 1 = 0}^{tp 1 maxz - 1} f ( P 1 t ) * Δ {t}_{P 1 maxz} - {\sum }_{P 2 = 0}^{tp 1 maxaxz - 1} f ( P 2 t ) * Δ {t}_{P 1 maxz}$$

The ratio of the difference thus obtained to A₁ can be recorded for each step:

$$( {A}_{1} - {A}_{2} ) : {A}_{1}$$

The time diagram shown on the left of FIG. 4B, representing instructed-step data allows that the appropriate step parameter or parameters can be stored, and the current steps can be compared to it or them. Expediently, therefore, the measurement and evaluation of a foot-ground interaction process is also performed with load-awareness on the big toe, storing the data obtained as a result, and taking into account the stored data for determining the existence or the extent of a risk of supination ankle injury. More preferably still, the exposure risk value or the protection value is determined with load-awareness on the big toe and also with spontaneous load on the big toe and determining the existence of a risk of supination ankle injury or the amount of said risk based on a comparison of the two obtained values, for example by calculating the ratio or difference of the two obtained values.

The existence of a risk of supination ankle injury can for example be determined by comparison to a threshold value, which threshold value can expediently be established empirically. The extent of supination ankle injury can for example be indicated in the form of a percentage. The indication can also be given—for example to the subject being examined—immediately after the foot-ground interaction process, but remote indication and temporally delayed alarms are also possible.

The time window between the time instant of the first pressure value measured in the first insole region 14 and the last measured maximum pressure value can be considered as the stance phase of a step consisting of initial contact, midstance, and terminal stance events, where:
P₁ₘₐₓ is the maximum (greatest) pressure value measured in the first insole region 14 in the stance phase of the step,
P₂ₘₐₓ is the maximum (greatest) pressure value measured in the first insole region 15 in the stance phase of the step,
t_{P1} is a time instant measured in the first insole region 14 with a certain pressure value, expediently recorded in milliseconds, where time is measured, starting from zero, at the time instant when the pressure appears in the first insole region 14,
t_{P1max} is the time instant of the maximum pressure value measured in the first insole region 14, recorded in milliseconds, where time is measured, starting from zero, at the time instant when the pressure first appears in the first insole region 14,
t_{P1maxz} is the last time instant, recorded in milliseconds, when the maximum pressure value is measured in the first insole region 14,
Δt_{P1maxz} denotes the time interval that lasts from the appearance of pressure force (P₁>0) in the first insole region 14 until the maximum pressure value (P₁ₘₐₓ) appears for the last time.
t_{P2} is a time instant measured in the second insole region 15 with a certain pressure value, recorded in milliseconds, where time is measured, starting from zero, at the time instant when the pressure first appears in the second insole region 15,
t_{P2max} is the time instant of the maximum pressure value measured in the second insole region 15, recorded in milliseconds, where time is measured, starting from zero, at the time instant when the pressure first appears in the second insole region 15,
the index “n” indicates a normal, i.e., non-instructed or spontaneous step, i.e., the stance phase thereof,
the index “i” indicates an instructed step, i.e., the stance phase thereof,
Δt_{P1>0→}t_{P2>0} is a time window, recorded in milliseconds, that starts from the time instant of the first non-zero pressure value in the first insole region 14 with the time value 0, and ends at the time instant of the first non-zero pressure value measured in the second insole region 15.

The area determined by plotting the pressure values recorded in the first insole region 14 between the first pressure value (P1>0, t_{P1>0}) and the last maximum pressure value (P₁ₘₐₓ, t_{P1maxz}) on the time axis can be interpreted as a “window of risk”.

The area determined by plotting the pressure values recorded in the second insole region 15 between the first pressure value (P2>0, t_{P2>0}) and the time instant t_{P1maxz} on the time axis can be interpreted as a window of protection.

The ratio of the area determined by plotting the pressure values recorded in the second insole region 15 between the first pressure value (P₂>0, t_{P2}>0) and the time instant t_{P1maxz} on the time axis and the area determined by plotting the pressure values recorded in the first insole region 14 between the first pressure value (P₁>0, t_{P1}>0) and the last maximum pressure value (P₁ₘₐₓ, t_{P1maxz}) on the time axis indicates, for each step, the protection ratio as a percentage:

$${A}_{2} : {A}_{1}$$

The area determined by plotting the pressure values recorded in the second insole region 15 between the first pressure value (P2>0, tP_{2>0}) and the time instant t_{P1maxz} on the time axis can be subtracted from the area determined by plotting the pressure values recorded in the first insole region 14 between the first pressure value (P1>0, t_{P1>0}) and the last maximum pressure value (P₁ₘₐₓ, t_{P1maxZ}) on the time axis: A₁-A₂

By dividing this value by the value of A₁: (A_{O2}-A_{I1}):A_{O2}
the obtained ratio gives the percentage of the absolute exposure risk, as detected for each step.

In the case of instructed steps (i) attention is drawn to the importance of putting load on the big toe in the midstance phase. If an increase of the exposure risk and a corresponding decrease of protection can be detected in the normal state (n) after recording the measured and calculated data for an instructed step, this can be considered a “loss of attention/focus” event. This state clearly involves a risk of supination ankle injury.

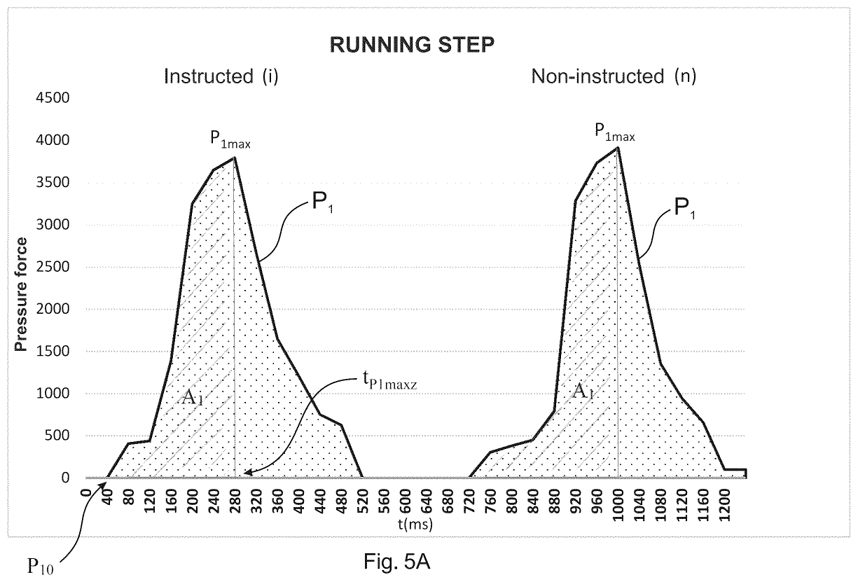

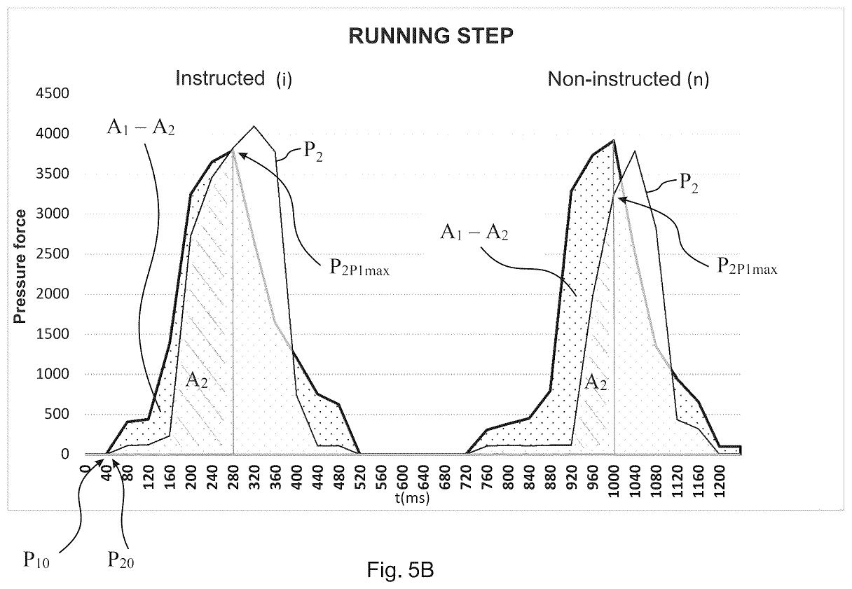

FIGS. 5A and 5B show experimental results recorded at a left foot during running in the same way as FIGS. 4A and 4B. The lines, areas, and value indications appearing in FIGS. 5A and 5B should be interpreted in the same way as in the case of FIGS. 4A and 4B.

The risk of injury increases from the time instant of detecting pressure in the first insole region 14 for the first time until the time instant t_{P1maxz}, the exposure risk being the greatest at the time instant t_{P1maxz}. After the time instant t_{P1maxz}, load on the lateral edge of the foot already decreases, so at this time there is no more risk of injury.

The risk is the greatest in case only A₁ has a non-zero value, i.e., weight is rolled only onto the lateral edge of the foot during the step, with no load being put on the big toe. The risk of injury decreases with an increasing load being put on the big toe during the step. The pressure that builds up in the second insole region 15 until the time instant t_{P1maxz} counteracts this risk. The degree of protection against the risk of injury has significance until the time instant t_{P1maxz}.

The ratio of the protection values measured during instructed (i) walking, running, jumping, and landing to the normal values (n) constitutes the instructed protection value for each step:

$${( {A}_{2} : {A}_{1} )}_{i} : {( {A}_{2} : {A}_{1} )}_{n}$$

In addition to indicating the risk of injury, the invention has several other advantages in comparison with prior art methods and devices. By informing the user of “improper” movement sequences, the device and method according to the invention helps develop and maintain appropriate biomechanical processes during gait and other movement types because it allows the user to apply corrections before the next gait/movement cycle. The invention also helps the user to become aware of the proper gait/movement patterns, namely, of the need to properly roll the body weight onto the foot, and especially of the proper use of the big toe.

The invention is not limited to the preferred embodiments described as examples above, but further variants are possible within the scope of protection determined by the claims. More than one sensor can also be arranged in each of the first and second insole regions 14, 15, and their signals can also be combined with each other; such a solution is also conceivable wherein sensors of different types are arranged in the same insole region 14, 15 or outside the regions to support the measurements from various sources. The insole 10 of the device according to the invention can be removable, or can be implemented to be permanently secured in the shoe after insertion; the latter case also includes such embodiments wherein the device according to the invention is integrated in the shoe. Pressure force values recorded outside the first and second time intervals according to the invention can also be considered or utilised for the purposes of the invention.

1. A method for indicating a risk of supination ankle injury, the method comprising applying an insole (10) placed in a shoe, characterised by comprising the steps of
receiving a first signal value sequence (P₁) provided by a first pressure force sensor (21) arranged in a first insole region (14) between three-fifths and four-fifths of a longitudinal expanse extending from a posterior end (11) of the insole (10) to an anterior end (12) of the insole (10) and laterally outward from a centreline (13) of said longitudinal expanse in a transversal direction perpendicular to the longitudinal direction,
receiving a second signal value sequence (P₂) provided by a second pressure force sensor (22) arranged in a second insole region (15) between three-fifths and the anterior end (12) of the longitudinal expanse extending from the posterior end (11) of the insole (10) to the anterior end (12) of the insole (10) and laterally inward from the centreline (13) of said longitudinal expanse in the transversal direction perpendicular to the longitudinal direction,
and, during a foot-ground interaction process
calculating a first aggregate value utilising first signal value sequence (P₁) values within a first time interval determined by a value of the first signal value sequence (P₁) indicating an appearance of a pressure force (P₁₀) and a value of the first signal value sequence (P₁) indicating maximum pressure force (P₁ₘₐₓ),
calculating a second aggregate value utilising second signal value sequence (P₂) values within a second time interval determined by a value of the second signal value sequence (P₂) indicating an appearance of a pressure force (P₂₀) and a value (P_{2P1max}) of the second signal value sequence (P₂) taken at the time instant corresponding to the time instant of the value (P₁ₘₐₓ) of the first signal value sequence (P₁) indicating maximum pressure force, and
determining an existence of a risk of supination ankle injury, or an extent of said risk, based on a comparison of the first aggregate value and the second aggregate value.

2. The method according to claim 1, characterised by considering, in case the values of the signal value sequence (P₁) reach the value (P₁ₘₐₓ) corresponding to the maximum pressure force more than once, the time instant corresponding to the last of these values (P_{1maxz}) as the endpoint of the first time interval.

3. The method according to claim 1, characterised in that the calculation of the first and second aggregate values comprises summation or integration along the time axis.

4. The method according to claim 1, characterised by determining the existence of a risk of supination ankle injury, or the extent of said risk, based on the ratio and/or difference of the first aggregate value and the second aggregate value.

5. The method according to claim 4, characterised by determining an exposure risk value by subtracting the second aggregate value from the first aggregate value, and dividing the difference thus obtained by the first aggregate value.

6. The method according to claim 5, characterised by determining a protection value by dividing the second aggregate value by the first aggregate value.

7. The method according to claim 1, characterised by performing a measurement and an evaluation of a foot-ground interaction process also with load-awareness on the big toe, storing the data obtained as a result, and taking into account the stored data for determining the existence or the extent of a risk of supination ankle injury.

8. The method according to claim 7, characterised by determining the exposure risk value or the protection value with load-awareness on the big toe and also with spontaneous load on the big toe, and determining the existence of a risk of supination ankle injury or the amount of said risk based on a comparison of the two obtained values.

9. The method according to claim 8, characterised by determining the existence of a risk of supination ankle injury, or the extent of said risk, based on the ratio and/or difference of said two obtained values.

10. The method according to claim 1, characterised by applying a second pressure force sensor (22) arranged in a portion of the second insole region (15) between four-fifths and the anterior end (12) of a longitudinal expanse extending from the posterior end (11) of the insole (10) to the anterior end (12) of the insole (10).

11. The method according to claim 1, characterised by indicating the existence of a risk of supination ankle injury or the extent of said risk surpassing a threshold value immediately following the foot-ground interaction process.

12. The method according to claim 1, characterised in that the foot-ground interaction process is a part of a walking, running, or jumping process, or a sporting activity.

13. A device for indicating a risk of supination ankle injury comprising an insole (10) placeable in a shoe, characterised by further comprising
a first pressure force sensor (21) arranged in a first insole region (14) between three-fifths and four-fifths of a longitudinal expanse extending from a posterior end (11) of the insole (10) to an anterior end (12) of the insole (10) and laterally outward from a centreline (13) of said longitudinal expanse in a transversal direction perpendicular to the longitudinal direction,
a second pressure force sensor (22) arranged inside a second insole region (15) between three-fifths and the anterior end (12) of the longitudinal expanse extending from the posterior end (11) of the insole (10) to the anterior end (12) of the insole (10) and laterally inward from the centreline (13) of said longitudinal expanse in the transversal direction perpendicular to the longitudinal direction, and
a control unit (23) adapted for receiving and processing a first signal value sequence (P₁) provided by the first pressure force sensor (21) and a second signal value sequence (P₂) provided by the second pressure force sensor (22) and being programmed to carry out the method according to claim 1.

14. The device according to claim 13, characterised in that the second pressure force sensor (22) is arranged in a portion of the second insole region (15) between four-fifths and the anterior end (12) of a longitudinal expanse extending from the posterior end (11) of the insole (10) to the anterior end (12) of the insole (10).

15. The device according to claim 13, characterised in that the first pressure force sensor (21) and/or the second pressure force sensor (22) is affixed onto the insole (10) or is fixed within the insole (10).

16. The device according to claim 13, characterised in that the insole (10) further comprises a dorsum-of-foot portion, the first pressure force sensor (21) and/or the second pressure force sensor (22) being affixed on the dorsum-of-foot portion or being fixed within the dorsum-of-foot portion.

17. The device according to claim 13, characterised in that the control unit (23) is arranged in the insole (10).

18. The device according to claim 13, characterised in that the control unit (23) is at least partially implemented as an external unit, and the device comprises a wired or wireless communication channel (24) between the external unit and other portions of the device.

19. The device according to claim 13, characterised in that it comprises an indicator device (25) adapted for indicating to a user wearing a shoe provided with the insole (10) the existence of a risk of supination ankle injury or the extent of said risk surpassing a threshold value.

20. The device according to claim 19, characterised in that the indicator device (25) is a haptic, audio, or visual indicator device arranged within the insole (10), on the user's body, or in the vicinity of the user.

21. A shoe for indicating a risk of supination ankle injury, comprising a device according to claim 13 removably or permanently arranged within the shoe.
